(19) United States
(12) Patent Application Publication
(10) Pub. No.: US 2011/0098326 A1
(43) Pub. Date: Apr. 28, 2011

(54) 2-FLUOROTHIAZOLE DERIVATIVES USEFUL AS IMAGING AGENTS; METHODS OF SYNTHESIS, AND METHODS OF USE

(76) Inventors: Victor W. Pike, Bethesda, MD (US); Fabrice G. Simeon, Washington, DC (US)

(21) Appl. No.: 12/605,467

(22) Filed: Oct. 26, 2009

(51) Int. Cl. A61K 31/426; C07D 277/32; G01N 33/53; A61P 25/28; A61P 25/30; A61P 25/24; A61P 25/22

(52) U.S. Cl. 514/365; 548/202; 548/203; 435/7.22

(57) ABSTRACT

Novel ¹⁸F-labeled thiazole derivatives useful for imaging of metabotropic glutamate subtype 5 receptors (mGluR5) in living mammalian brain are disclosed herein. Also disclosed herein is a synthetic method for making the claimed thiazole derivatives under thermal heating or microwave conditions for aryl thioethers that provides the compounds in high yield. Imaging methods in which the claimed ¹⁸F-labeled thiazole derivatives are used as imaging agents are also disclosed. Halogen substituted thiazole derivative disclosed herein are also useful as therapeutic agents. Methods of treating mGluR5 mediated disorders with certain halogen substituted thiazole derivatives are disclosed.

### STATEMENT OF GOVERNMENT SUPPORT

This invention was made in part with government support from the National Institute of Health. The government has certain rights in this invention.

### BACKGROUND

Metabotropic glutamate receptors are G-coupled protein receptors that modulate intracellular second messenger systems. Agonist binding to the metabotropic glutamate receptor subtype 5 (mGluR5) stimulates phospholipase C, which results in phosphoinositide hydrolysis and mobilization of intracellular calcium. Animal studies suggest mGluR5 is impaired or otherwise abnormal (e.g., with regard to density and distribution) in several human disorders, including anxiety, schizophrenia, substance abuse, and fragile X syndrome. In response to these animal studies, several drugs that modulate mGluR5 function are being developed. The drug target on mGluR5 is distinct from the glutamate binding site. Instead, the drug target is an allosteric (or noncompetitive) site that can either increase or decrease function via positive or negative allosteric modulation, respectively.

Thus far, drugs prepared as effective modulators of the allosteric site of the mGluR5 have not proven useful as therapeutic agents. Radiolabeled agents specific to the allosteric site of mGluR5 are needed to elucidate the activity of mGluR5 modulators in living brain by imaging with positron emission tomography (PET) or other modalities.

The inventors hereof have previously reported an effective mGluR5 PET imaging agent, 3-fluoro-5-[[2-([¹⁸F]fluoromethyl)thiazol-4-yl]ethynyl]benzonitrile ([¹⁸F]SP203). This compound is rapidly defluorinated in monkey and rat, although defluorination is slower or negligible in human subjects. In brain PET scans, radiodefluorination hampers quantification of brain mGluR5 density in non-human subjects since ¹⁸F-labeled fluoride ion accumulates in nearby bone (e.g. skull), leading to ‘spillover of radioactivity’ through the partial volume effect. Thus there remains a need for an effective mGluR5 allosteric site modulator that can be radiolabeled for use in imaging animals and human subjects with PET or other brain imaging techniques. The present 2-fluorothiazole compounds, compositions, and methods fulfill this need and provide additional advantages described herein.

### SUMMARY

Described herein are 2-fluorothiazole derivatives (including radiolabeled 2-fluorothiazole derivatives useful as labeled imaging agents), their methods of manufacture, compositions containing the 2-fluorothiazole derivatives, and methods of use of both the 2-fluorothiazole derivatives and compositions thereof. Thus in a first aspect, a compound of Formula I and IA are provided.

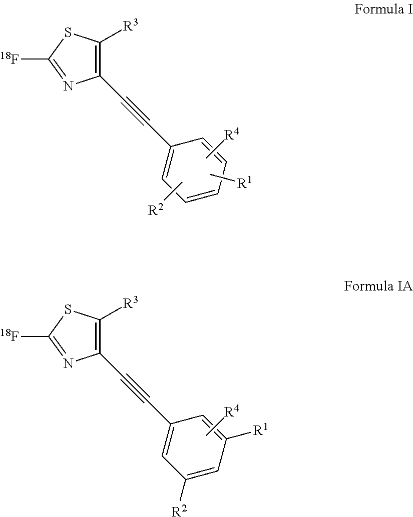

Within Formula I and Formula IA the variables R¹ to R⁴ carry the following definitions.

R¹ is hydrogen, cyano, C₁-C₂alkylnitrile, hydroxyl, C₁-C₂alkyl, aryl, C₁-C₂alkoxy, halogen, trifluoromethyl, or trifluoromethoxy;

R² is hydrogen, cyano, C₁-C₂alkylnitrile, hydroxyl, C₁-C₂alkyl, aryl, C₁-C₂alkoxy, halogen, trifluoromethyl, or trifluoromethoxy;

R³ is hydrogen, halogen, methyl, or methoxy; and

R⁴ is 0 to 3 substituents independently chosen from halogen, methyl, and methoxy.

A method of making a compound of Formula I, comprises heating a compound of the formula

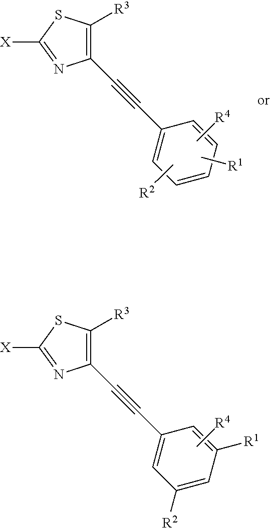

where X is a halogen radical selected from chloro, bromo, and iodo, or X is nitro, trimethylammonium, or X is an aryliodonium entity (ArI⁺), where Ar may be Ph or 2-thienyl or substituted Ph, especially methoxy substituted, or 2-thienyl or substituted 2-thienyl, especially methyl-substituted;

with [¹⁸F]F⁻—K⁺-4,7,13,16,21,24-hexaoxa-1,10-diazabicyclo[8.8.8]hexacosane (Kryptofix® 222) or [¹⁸F]F⁻—K⁺-1,4,7,10,13,16-hexaoxacyclooctadecane (18-crown-6) in an organic solvent under microwave or thermal conditions, or with [¹⁸F]F⁻-Cs⁺, in the presence or absence of either K⁺-Kryptofix® 222 or 18-crown-6, or with ¹⁸F⁻ in the presence of tetra-alkylammonium (R₄N⁺_{), where R is preferably Et, n-Bu or t-Bu.}

Also included herein is a method of making a compound of Formula II, or Formula IIA, respectively

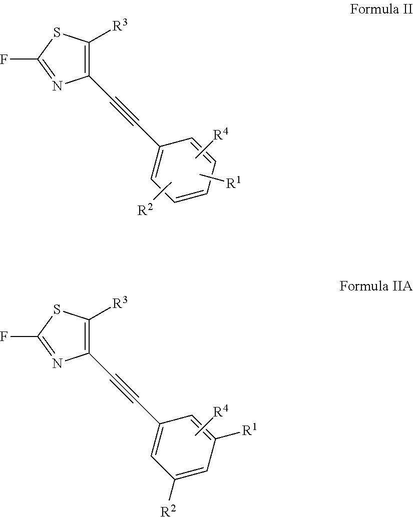

comprising contacting a compound of Formula III, or Formula IIIA, respectively,

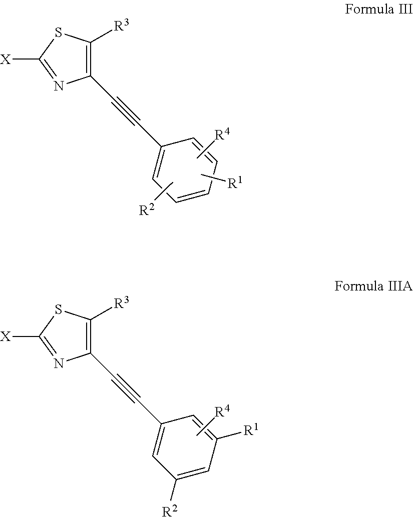

where X is a halogen radical selected from chloro, bromo, and iodo; or X is nitro, trimethylammonium, or X is an aryliodonium entity (ArI⁺), where Ar may be Ph or 2-thienyl or substituted Ph, especially methoxy substituted, or 2-thienyl or substituted 2-thienyl, especially methyl-substituted; with KF, CsF or a KF-AgF mixture in the presence of either Kryptofix® 222 or 18-crown-6, in an organic solvent under microwave or thermal conditions to provide a compound of Formula II.

In another aspect, a method of imaging mGluR5 in a mammal comprises administering a compound of Formula I to the mammal and imaging portions of the mammal where mGluR5 occur.

An in vitro method of imaging mGluR5 comprises contacting a sample containing mGluR5 with a compound of Formula I, removing the unbound compound from the sample, and detecting the bound compound in the sample.

In still another aspect, a pharmaceutical composition comprises a compound of Formula II together with at least one pharmaceutically acceptable carrier.

A method of treating an mGluR5 modulated disorder comprises administering a compound of Formula II to a patient having an mGluR5 mediated disorder. Such mGluR5 mediated disorders include, but are not limited to, autism, schizophrenia, Alzheimer's disease, anxiety, depression, drug addiction, and fragile X syndrome.

### DETAILED DISCUSSION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising”, “having”, “including”, and “containing” are to be construed as open-ended terms (i.e., meaning “including, but not limited to”). Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Formula I comprises all subformulae thereof, including Formula IA. Likewise Formula II comprises all subformulae thereof, including Formula IIA, and Formula III, comprises all subformulae thereof, including Formula IIIA.

All compounds are understood to include all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C. Isotopes particularly include radioisotopes, for example radioisotopes of iodine include ¹²³I, ¹²⁴I, and ¹²⁵I.

The term “substituted” means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

“Alkyl” includes both branched and straight chain saturated aliphatic hydrocarbon groups, having the specified number of carbon atoms. The term C₁-C₂alkyl as used herein means an alkyl group having from 1 to about 2 carbon atoms, e.g., methyl and ethyl, respectively.

“Alkylnitrile” is an alkyl group, as defined above, with the indicated number of carbon atoms substituted with a cyano (C≡N) group. The alkylnitrile is attached to the group it substitutes through by a covalent bond to its alkyl portion.

“Alkoxy” means an alkyl group, as defined above, with the indicated number of carbon atoms attached via an oxygen bridge.

“Aryl” is an aromatic group containing only carbon in the aromatic ring or rings. Certain aryl groups contain 1 to 2 separate, fused, or pendant rings and from 6 to about 12 ring atoms in total, without heteroatoms as ring members. Such aryl groups may be further substituted with carbon or non-carbon atoms or groups. Substituents include hydroxyl, amino, cyano, nitro, halogen, C₁-C₂alkyl, and C₁-C₂alkoxy. “Halo” or “halogen” means fluoro, chloro, bromo, or iodo.

“Pharmaceutical compositions” means compositions comprising at least one active agent, such as a compound or salt of the invention, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

“Providing” means giving, administering, selling, distributing, transferring (for profit or not), manufacturing, compounding, or dispensing.

“Treatment” or “treating” includes providing an active compound in an amount sufficient to: (a) prevent a disease or a symptom of a disease from occurring in a patient who may be predisposed to the disease but has not yet been diagnosed as having it (e.g., including diseases that may be associated with or caused by a primary disease; (b) inhibit the disease, i.e., arrest its development; and (c) relieve the disease, i.e., cause regression of the disease.

A “therapeutically effective amount” of a pharmaceutical composition means an amount effective, when administered to a patient, to provide a therapeutic benefit such as an amelioration of symptoms, e.g., an amount effective to decrease the symptoms of an mGluR5 mediated disorder. For example, in a patient having depression an effective amount would be an amount that results in any statistically significant improvement in the patients HAMD score upon treatment.

A significant change is any detectable change that is statistically significant in a standard parametric test of statistical significance such as Student's T-test, where p<0.05.

### Chemical

Compounds of Formula I are useful for imaging mGluR5, for example by PET and SPECT (Single Photon Emission Computed Tomography) imaging of mGluR5. The labeled compounds disclosed herein exhibit high binding affinity and specificity for mGluR5, with low non-specific binding for the non-target tissues or cells. Most previously reported ¹⁸F-labeled 2-fluoromethyl thiazole compounds exhibited high radiodefluorination in some mammalian species limiting the utility of these compounds. Direct fluorination at the thiazole 2-position is believed to minimize or abolish radiodefluorination.

In addition to compounds of Formula I, II, and III described above, specific 2-fluorothiazole derivatives include the compounds of Formula I, II, and III in which the variables have the meanings set forth below, or example compounds of Formula I, II, and III in which any of the following conditions are met:
(i) R³ is hydrogen and R⁴ is 0 substituents (absent).
(ii) R¹ is hydrogen, halogen, or cyano, C₁-C₂alkylnitrile; and R² is hydrogen, halogen, cyano or C₁-C₂alkylnitrile.
(iii) R¹ is hydrogen or fluoro; and R² is hydrogen or cyano.
(iv) R¹ is hydrogen or fluoro; and R² is hydrogen or cyano.
(v) R¹ and R² are both hydrogen.
(vi) R¹ is hydrogen and R² is cyano.
(vii) R¹ is fluoro and R² is cyano.

Also included are all radioisotopes of Formula I, II, and III. For example radioiodinated compounds in which a halogen at R¹, R², R³ or X is ¹²³I, ¹²⁴I, ¹²⁵I are included herein. These compounds can be prepared by the methods given herein, with routine modifications that are readily apparent to those of skill in the art of organic chemistry synthesis.

Any of the above conditions, (i) to (vii), may be combined so long as an isolatable compound results. For example, the radiolabeled 2-fluorothiazole derivatives include a compound of Formula I in which condition (i) and (vii) are combined, that is, R³ is hydrogen and R⁴ is 0 substituents, and R¹ is fluoro and R² is cyano, i.e., a compound of the formula

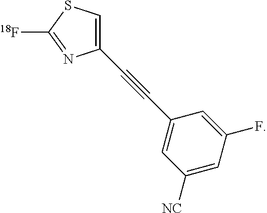

Another embodiment provides method of imaging mGluR5 in a mammal by administering a compound of Formula I to the mammal and imaging portions of the mammal where mGluR5 occurs. In certain embodiments the mammal is a mouse, a rat, a monkey or a human.

Imaging methods include in vitro methods such as contacting a sample containing mGluR5 with a compound of Formula I, removing the unbound compound from the sample, and detecting the bound compound in the sample. Typically the sample will be a brain section prepared for autoradiography, and the detecting is via autoradiography.

### Synthetic Methods

In still another aspect, methods for making compounds of Formulas I and II and methods for preparing intermediates of Formulas I and II are described.

Also provided is a method of making a compound of Formula I comprising heating a compound of the formula

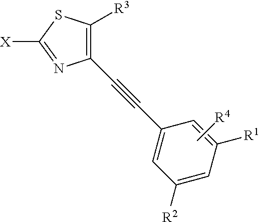

where X is a halogen radical selected from chloro, bromo, and iodo; or X is nitro, trimethylammonium, or X is an aryliodonium entity (ArI⁺), where Ar may be Ph or 2-thienyl or substituted Ph, especially methoxy substituted, or 2-thienyl or substituted 2-thienyl, especially methyl-substituted;

with [¹⁸F]F⁻—K⁺-Kryptofix® 222 or [¹⁸F]F⁻—K⁺-18-Crown-6 in an organic solvent under microwave or thermal heating conditions to provide a compound of Formula I, or with [¹⁸F]FCs, in the presence or absence of K⁺-4,7,13,16,21,24-hexaoxa-1,10-diazabicyclo[8.8.8]hexacosane, or with ¹⁸F⁻ in the presence of tetra-alkylammonium (R₄N⁺), where R is preferably Et, n-Bu or t-Bu.

[¹⁸F]F⁻—K⁺-4,7,13,16,21,24-hexaoxa-1,10-diazabicyclo[8.8.8]hexacosane is also known as a [¹⁸F]F⁻—K⁺-Kryptofix® 222 complex. (Kryptofix is a registered trademark of Merck KGAA Ltd.). The [¹⁸F]F⁻—K⁺-Kryptofix® 222 complex may be prepared, for example, by adding an aqueous solution of potassium carbonate and Kryptofix® 222 to an aqueous solution containing the [¹⁸F]fluoride ion. The [¹⁸F]F⁻—K⁺-Kryptofix® 222 complex can be isolated by drying the complex and dissolution in acetonitrile (MeCN) or other similarly polar solvent such as ethanol, methanol, or dioxane. Other well-known variants of this procedure may be used to produce the same complex.

Radiofluorination is effected, for example, by heating the precursor compound with [¹⁸F]F⁻—K⁺-Kryptofix® 222 in a non-polar solvent such as MeCN, dimethyl formamide (DMF), dimethyl sulfoxide (DMSO) or other nonpolar solvent, under microwave irradiation or thermal conditions. When microwave irradiation is used the conditions are about 30 to 90W, (from about 80 to about 150° C.), for about 2 minutes to about 30 minutes, or more commonly about 5 minutes to about 10 minutes.

A method of preparing a compound of Formula II comprises contacting a compound of the formula

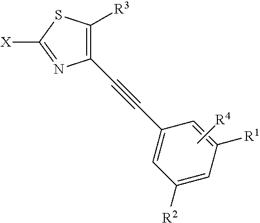

wherein X is a halogen radical selected from chloro, bromo, iodo or nitro; with KF, CsF or a KF-AgF mixture in an organic solvent, such as DMSO, MeCN, DMF, butanol, with Kryptofix® 222 or 18-Crown-6 under microwave conditions or thermal heating to provide a compound of Formula II. The microwave conditions include microwave irradiation at about 50 W, (about 10 minutes at about 130° C.).

In the above methods the variables R¹ to R⁴ may carry any of the values set forth for these variables in conditions (i) to (vii) in the Chemical Description section.

In one aspect, the intermediate

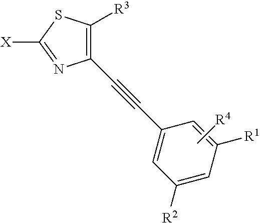

is provided by halogenating a compound of the formula

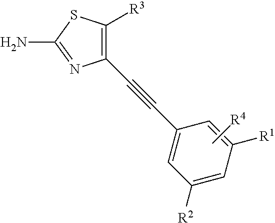

with

(i) CuX¹, where X¹ is chloro, bromo, or iodo, in the presence of n-butyl nitrite; or

(ii) alumina-KCuBr₂ (a preparation of KBr and CuBr on neutral alumina). In one embodiment, the 2-amino compound is dissolved in a polar solvent, such as acetonitrile at room temperature. The compound is heated in the presence of a nitrite (such as about 1 to 2 equivalents of n-butyl nitrite, tert-butyl nitrite or amyl nitrite) and alumina-KCuX₂ or CuX. The reaction proceeds at room temperature (with alumina complex) for 24 hours or proceeds at 40-85° C. (with copper salt) for about 10 minutes to about 1 hour (SCHEME 1).

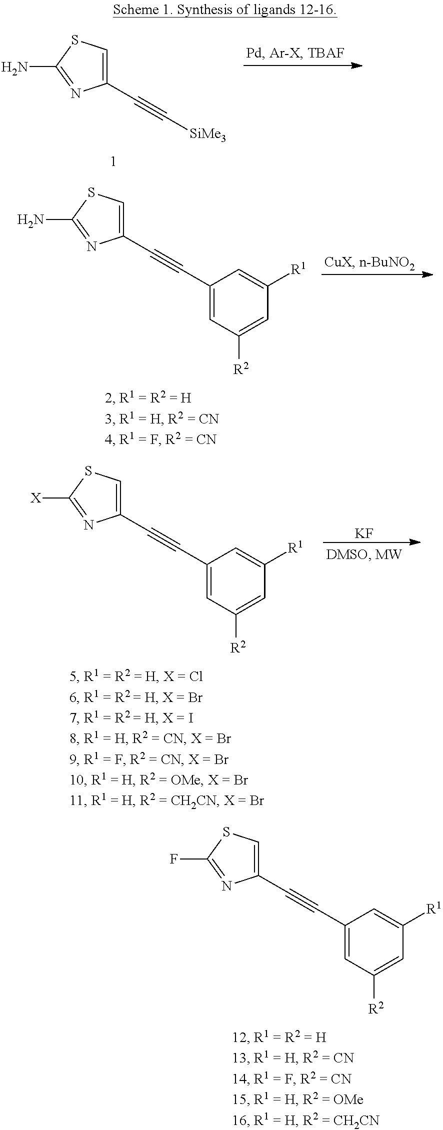

In another embodiment, the chlorothiazole derivatives of type (5a to 5e), were directly prepared by Sonogashira coupling of (17) with the appropriate haloarenes (SCHEME 2).

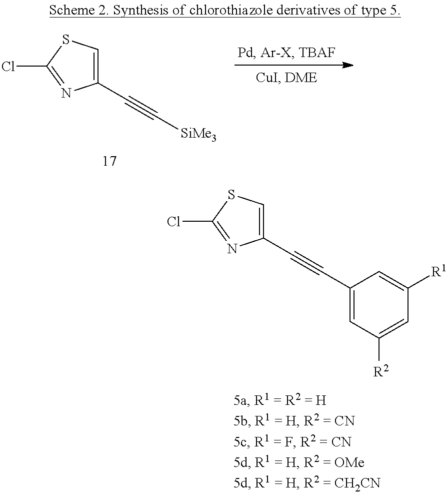

The preferred route to prepare radiolabeled compounds [¹⁸F]12-16 is described later in Scheme 4 (example 1). However, in another aspect of this invention, an alternate synthetic route, shown in SCHEME 3, below, was designed to produce radiolabeled compounds [¹⁸F]12-16. In this case, compound 17 was reacted with tetrabutylammonium fluoride (TBAF) in THF at room temperature to give the 2-chloro-4-ethynyl-1,3-thiazole 18.

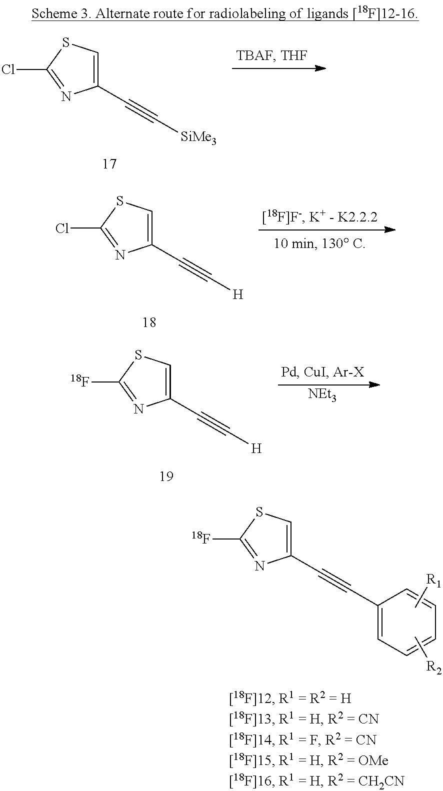

Acetylene 18 can be conveniently labeled by reaction with [¹⁸F]fluoride ion in the presence of kryptofix® 222 or ether 18-crown-6 to give the radiosynthon 19 (Scheme 3). Sonogashira cross-coupling of 19 with the appropriate aryl halide can produce [¹⁸F]12-16.

### Pharmaceutical Preparations

The 2-fluorothiazole derivatives can be administered as the neat chemical, but are preferably administered as a pharmaceutical composition, for example a pharmaceutical formulation comprising a 2-fluorothiazole derivative or pharmaceutically acceptable salt thereof, together with at least one pharmaceutically acceptable carrier.

The 2-fluorothiazole derivatives may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorings, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active and/or inactive agents may be included in the pharmaceutical compositions, provided that such agents do not substantially interfere with the activity of the compounds used in the pharmaceutical compositions.

Pharmaceutical compositions formulated for oral administration are often preferred. These compositions contain between 0.1 and 99 weight % (wt. %) of a 2-fluorothiazole derivative and usually at least about 5 wt. % of a 2-fluorothiazole derivative, particular, a 2-fluorothiazole derivative of formula (II). Some embodiments contain from about 25 wt. % to about 50 wt. % or from about 5 wt. % to about 75 wt. % of the 2-fluorothiazole derivative.

### Treatment Methods

The pharmaceutical compositions disclosed herein are useful for treating mGluR5 mediated disorders in patients. An effective amount of a pharmaceutical composition may be an amount sufficient to (a) prevent an mGluR5 mediated disorder or a symptom of an mGluR5 mediated disorder from occurring in a patient who may be predisposed to an mGluR5 mediated disorder but who has not yet been diagnosed as having it; (b) inhibit the progression of an mGluR5 mediated disorder; and (c) cause a regression of the mGluR5 mediated disorder.

An effective amount of a compound or pharmaceutical composition described herein will also provide a sufficient concentration of a 2-fluorothiazole derivative when administered to a patient. A sufficient concentration is a concentration of the compound in the patient's body necessary to prevent or combat the disorder. Such an amount may be ascertained experimentally, for example by assaying blood concentration of the compound, or theoretically, by calculating bioavailability. The amount of an active agent sufficient to modulate mGluR5 in vivo may be determined in vitro with a conventional assay for mGluR5 occupancy, which is described below in the examples.

Methods of treatment include providing certain dosage amounts of a 2-fluorothiazole derivative to a patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula II are provided daily to a patient. Frequency of dosage may also vary depending on the compound used and the particular disease treated. However, for treatment of most mGluR5 mediated disorders, a dosage regimen of 4 times daily or less is preferred and a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### EXAMPLES

### Example 1

### Synthesis of Radiolabeled 2-Fluorothiazole Derivatives

The method of making radiolabeled 2-fluorothiazole derivatives is depicted in Scheme 3, below.

Halo precursors (5-9) for radiolabeling were prepared by Sonogashira cross-coupling of 1 with the appropriate haloarenes followed by regioselective halogenation with CuX (X═Cl, Br, I for compounds 5-7, respectively; X═Br for compounds 8 and 9) in the presence of n-butyl nitrite (Schemes 1 and 2). The corresponding fluoro analogs (10-12) were obtained in 25-40% yield by treating the bromides (6, 8 or 9) with KF in DMSO under microwave irradiation at 150 W (10 min, 150° C.). Use of a KF-AgF mixture improved yields to 75%. An aqueous solution (100 μL) of potassium carbonate (0.5 mg) and Kryptofix® 222 (5 mg) was added to aqueous cyclotron-produced [¹⁸F]fluoride ion (150-250 μL), and dried by three cycles of acetonitrile addition evaporation under a nitrogen stream. Each halo precursor (5-9) (about 1 mg) was heated with the generated [¹⁸F]F⁻—K⁺-Kryptofix® 222 complex for different times and temperatures in MeCN or DMSO (750 μL) under microwave irradiation at 90 W (80 to 150° C.). Radioactive products were purified on a semi-preparative size Luna C18 column being eluted with acetonitrile: aq. 10 mM ammonium formate (60: 40, v/v) at 4.5 mL/min, and then analyzed by coinjection with reference ligand and LC-MS analysis of carrier.

Treatment of 5, 6 or 7 with [¹⁸F]fluoride ion in DMSO under microwave irradiation (5-10 min, 150° C., 90 W) produced [¹⁸F]12 ([¹⁸F]labeled 2-fluoro-4-(phenylethynyl)thiazole) in 19, 16 or 3% decay-corrected radiochemical yield (RCY), respectively. Radiofluorination of 6 at 130° C. in DMSO under argon for 10 min. gave [¹⁸F]12 in much higher RCY (52%). The radiofluorination of 9 under mild conditions (MeCN, 80° C., 30 min) gave ¹⁸F-labeled 3-fluoro-5-((2-fluorothiazol-4-yl)ethynyl)benzonitrile (label at 2-fluoro) in only 4% RCY, but under harsher conditions (DMSO, 130° C., 10 min) gave high RCY (86%). Under the latter conditions, [¹⁸F]13 (¹⁸F-labeled (5-((2-fluorothiazol-4-yl) ethynyl)benzonitrile)) was produced from 8 in 37% RCY.

The reaction of 2-halo-substituted 1,3-thiazoles with [¹⁸F]fluoride ion gave the [¹⁸F]2-fluoro analogs in high RCYs.

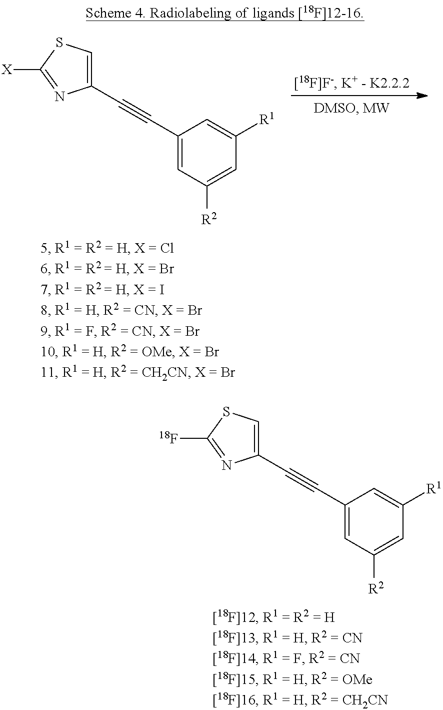

### Example 2

### In Vitro Receptor Binding to Rat Brain Membranes

The affinity of test compounds for rat brain membranes is determined by displacement of [³H]MPEPy binding to rat brain membranes. Whole rat brain, minus cerebellum and brainstem, is homogenized (1:10 w/v) in ice-cold assay buffer (50 mM Tris-HCl-1.1% saline buffer, pH 7.5). The homogenate is centrifuged at 800×g for 10 min at 4° C., and the resulting pellet is resuspended in buffer (6.25 mg original wet weight per mL) and stored at −70° C. until analysis. Immediately before analysis, membranes are resuspended in assay buffer (0.9% NaCl containing 50 mM Tris-HCl) to give a final assay concentration of 5 mg/800 μL. Each competing ligand (10 mM in EtOH) is added in a volume of 100 μL to give a final concentration in the range of 0.01 nM to 1 mM along with 100 μL of [³H]MPEPy. The incubation is initiated by adding membranes (800 μL) to constitute a total assay volume of 1 mL and is allowed to proceed for 1 hour at 22° C. The assay is terminated by rapid filtration over Whatman GF/B filters that had been presoaked in poly(ethyleneimine) (0.5%) and then three washes with ice-cold saline (0.9% NaCl; 3 mL). Radioactivity is determined by liquid scintillation counting.

### Example 3

### In Vitro Binding to Glutamate Receptors

The assay buffer is Standard Binding Buffer (50 mM Tris-HCl, 10 mM MgCl₂, 0.1 mM EDTA, pH 7.4) and the membrane fraction source is transiently or stably transfected cell lines (e.g., HEK293, COS, CHO, NIH3T3)

A solution of the compound to be tested is prepared as a 1-mg/ml stock in the Standard Binding Buffer or DMSO according to its solubility. A similar stock of reference compound (positive control) is also prepared. Eleven dilutions (5× assay concentration) of the test and reference (MTEP) compounds are prepared in Standard Binding Buffer by serial dilution: 0.05 nM, 0.5 nM, 1.5 nM, 5 nM, 15 nM, 50 nM, 150 nM, 500 nM, 1.5 μM, 5 μM, 50 μM (thus, the corresponding assay concentrations span from 10 μM to 10 μM and include semilog points in the range where high-to-moderate affinity ligands compete with radioligand for binding sites).

Radioligand ([³H]MPEP) is diluted to 4.5 nM (five times the assay concentration) in Standard Binding Buffer.

Aliquots (50 μL) of radioligand are dispensed into the wells of a 96-well plate containing 100 μl of Standard Binding Buffer. Then, duplicate 50-μL aliquots of the test and reference compound dilutions are added.

Finally, crude membrane fractions of cells expressing recombinant target (prepared from 10-cm plates by harvesting PBS-rinsed monolayers, resuspending and lysing in chilled, hypotonic 50 mM Tris-HCl, pH 7.4, centrifuging at 20,000×g, decanting the supernatant and storing at −80° C.; typically, one 10-cm plate provides sufficient material for 24 wells) are resuspended in 3 mL of chilled Standard Binding Buffer and homogenized by several passages through a 26 gauge needle, then 50 μL are dispensed into each well.

The 250-4, reactions are incubated at room temperature and shielded from light (to prevent photolysis of light-sensitive ligands) for 1 hour, then harvested by rapid filtration onto Whatman GF/B glass fiber filters pre-soaked with 0.3% polyethyleneimine using a 96-well Brandel harvester. Four rapid 500-μL washes are performed with chilled Standard Binding Buffer to reduce non-specific binding. Filters are placed in 6-mL scintillation tubes and allowed to dry overnight. The next day, 4 mL of EcoScint scintillation cocktail (National Diagnostics) are added to each tube. The tubes are capped, labeled, and counted by liquid scintillation counting. For higher throughput assays, bound radioactivity is harvested onto 0.3% polyethyleneimine-treated, 96-well filter mats using a 96-well Filtermate harvester. The filter mats are dried, then scintillant is melted onto the filters and the radioactivity retained on the filters is counted in a Microbeta scintillation counter.

Raw data (dpm) representing total radioligand binding (i.e., specific+non-specific binding) are plotted as a function of the logarithm of the molar concentration of the competitor (i.e., test or reference compound). Non-linear regression of the normalized (i.e., percent radioligand binding compared to that observed in the absence of test or reference compound) raw data is performed in Prism 4.0 (GraphPad Software) using the built-in three parameter logistic model describing ligand competition binding to radioligand-labeled sites:

y=bottom+[(top-bottom)/(1+10×-log IC₅₀)] (Eqn. 1)

where bottom equals the residual radioligand binding measured in the presence of 10 μM reference compound (i.e., non-specific binding) and top equals the total radioligand binding observed in the absence of competitor. The log IC₅₀ (i.e., the log of the ligand concentration that reduces radioligand binding by 50%) is thus estimated from the data and used to obtain the K, by applying the Cheng-Prusoff approximation:

Kᵢ=IC₅₀/(1+[ligand]/K_{D}) (Eqn. 2)

where [ligand] equals the assay radioligand concentration and K_{D} equals the affinity constant of the radioligand for the target receptor.

Compounds 12-16 were tested against mGluR5 receptors and found to exhibit high affinity and high selectivity for this receptor. The affinity data is presented in Table 1. In Table 1 ** indicates a binding affinity of less than 150 nM and *** indicates a binding affinity of less than 50 nM.

TABLE 1

| Entry | R¹ | R² | Ki (nM) |
|---|---|---|---|
| 12 | H | H | ** |
| 13 | H | CN | *** |
| 14 | F | CN | *** |
| 15 | H | OMe | *** |
| 16 | H | CH₂CN | *** |

### Example 4

### HPLC Analysis of Stability of High Specific Radioactivity [¹⁸F] Test Compound in Rat Brain and Blood In Vitro

A blood sample (2 mL) is drawn from an anesthetized rat via cardiac puncture into a heparinized tube. Ice-cold (4° C.) heparinized saline (100 units/mL; 40 mL) is perfused through the left ventricle of the heart until perfusate, when leaving the opened right atrium, is clear of blood. The brain is excised and placed in ice-cold (4° C.) heparinized saline (5 mL) in an ice-cold glass tube of a tissue homogenizer (model 099C-K54; Glas-Col, Terre Haute, Ind.) and homogenized by three Teflon pestle plunges with a 5-min pause after each plunge. [¹⁸F-labeled] test compound (20 μCi) is added to the brain homogenate (5 mL) and then incubated at 37° C. in a reciprocating-shaker water bath (model 25; Precision Scientific, Chicago, Ill.) at 60 oscillations/min. Aliquots (50 n1) from the incubation mixture are removed at 5, 10, 15, 30, and 60 min, placed in acetonitrile (300 n1) that had been spiked with test compound, and then mixed. Potassium fluoride solution (0.5 M; 50 μl) is added, and the solution is mixed again. All samples are measured in a gamma counter and then centrifuged at 10,000×g for 1 minute. The supernatant liquids are analyzed by radio-HPLC. The precipitates are then counted in a gamma counter for calculating the recovery of radioactivity into the acetonitrile.

In a separate experiment, rat brain homogenates are incubated with [¹⁸F-labeled] compound for 1.5 hours, and then they are analyzed simultaneously on two chromatographic systems to separate and identify radiometabolites. One method is based on the use of a Shodex ODP2 HP-4E column (250×4.6 mm, 5 μm; Showa Denko America, Inc., New York, N.Y.). The Shodex column has mixed mode features (size exclusion and polymer-based stationary phase) that allow polar compounds, such as [¹⁸F]fluoride ion, to be retained when acetonitrile predominates in the mobile phase. Thus, the column is equilibrated with acetonitrile/100 mM ammonium formate [80:20 (v/v); pH 4.5] at 0.7 mL/min. All samples are deproteinized with acetonitrile and adjusted to a final acetonitrile composition of 80%. Up to 1 mL of the biological sample is injected onto the Shodex column. Potassium fluoride solution (>0.5 mM; 100 μL) is added to enhance separation of [¹⁸F]fluoride ion from unchanged [¹⁸F]test compound on the Shodex column.

For blood analysis, sample (2 mL) is mixed with ¹⁸F-labeled test compound (3.5 μCi) in a polypropylene tube (13×60 mm) and incubated at 37° C., as described above. A 50-μL aliquot of the radioactive blood is removed at 5, 10, 15, 30, and 60 min and placed in saline (1 ml) and centrifuged (1800×g; 1.5 min). The supernatant plasma saline is separated from the pelleted cells. The cell and plasma saline fractions are mixed first with acetonitrile (300 μL) that had been spiked with test compound and then with potassium fluoride solution (0.5 M; 50 μL). The samples are measured in a gamma counter and centrifuged (10,000×g; 1 min), and the supernatant liquids are injected onto radio-HPLC for analysis. The precipitates are then measured in a gamma counter to calculate recovery of radioactivity into the acetonitrile.

### Example 5

### Positron Emission Tomography

ANIMAL CARE AND USE. Experiments in rhesus monkeys (Macacca mulatto) are conducted according to the Guide for the Care and Use for Laboratory Animals. Rhesus monkeys (10 kg) are initially anesthetized with ketamine (10 mg/kg, i.m.) and then induced with propofol (5 mg/kg, i.v.), intubated, and respired with medical grade air. During the PET scanning sessions, anesthesia is maintained by continuous administration of isoflurane at 1.0-4.0% in oxygen via the endotracheal tube. Body temperature is maintained between 37 and 37.5° C. Electrocardiograph, heart, and respiration rates are also monitored throughout the experiment.

PET scans are performed in a high-resolution research tomograph (HRRT, Siemens/CPS, Knoxyille, Tenn.). Transmission scans are performed with a Cs point source following injection of 2 to 5 mCi of the PET radioligand. Images are reconstructed with attenuation and scatter correction using a list mode OSEM algorithm, resulting in an image resolution of 2.5 mm fwhm (full width at half maximum). Tomographic images are analyzed with PMOD 2.75 (PMOD Technologies Ltd, Adliswil, Switzerland). For each scan, a static PET image is obtained by summing the dynamic frames acquired during the acquisition. In a baseline experiment, [¹⁸F]test compound (3.99 mCi; specific radioactivity, 1.93 Ci/micromol) is injected intravenously into the monkey (14.7 kg) as a bolus in physiological saline (5 mL) containing ethanol (10%). For a pretreatment experiment on a separate day, MTEP (2-5 mg/kg) in physiological saline containing ethanol (10%) is injected intravenously into the same monkey at 15 min before injection of ¹⁸F-labeled test compound (3.82 mCi; specific radioactivity, 2.02 Ci/micromol).

Regions of interest are drawn on the coregistered images in striatum (caudate and putamen), thalamus, hippocampus, occipital cortex, cerebellum, and bone (mandible). These regions were transferred onto the dynamic scans to obtain the corresponding time-activity curves. Radioactivity was expressed as standardized uptake value (SUV), which normalizes for injected activity and body weight.

### Example 6

### MRI and Image Fusion

All monkeys have T1-weighted magnetic resonance imaging (TR/TE/x) 24 ms/3 ms/300), acquired on a 1.5-T GE Horizon Instrument (General Electric Medical Systems, Waukesha, Wis.). PET and NMR images are coregistered with SPM2 (Wellcome Department of Cognitive Neurology, London, U.K.).

### Example 7

### Emergence of Radiometabolites in Monkey Blood In Vivo

Heparinized blood samples (1 mL) are drawn at 1 min intervals for 5 min and then at 10, 25, 40, 60, 90, 120, and 180 min after injection of [¹⁸F]test compound (2.2. mCi) into an anesthetized monkey. They are placed immediately on ice to retard ex vivo metabolism.

Measured aliquots (by pipetting; about 1 mL) of blood are then centrifuged at 1800×g for 1.5 min and plasma separated. The cellular and the plasma fractions are then counted in a gamma-counter. The distribution of radioactivity is calculated for each sample. The total radioactivity in the plasma and the cells represents the amount in whole blood.

### Example 8

### Stability of [¹⁸F]Test Compound in Monkey and Human Whole Blood and Plasma In Vitro

In these experiments, the initial radiochemical purity of ¹⁸F-labeled test compound and subsequent measures of stability is determined by reverse phase chromatography on a Novapak C-18 column (100×8 mm; Waters Corp., Milford, Mass.) using a Radial-Pak compression module RCM-11 with a sentry precolumn and a mobile phase of MeOH—H₂O-Et₃N (70:30:0.1) at a flow rate of 2.0 mL/min.

To test for the stability of ¹⁸Flabeled test compound in monkey whole blood, anticoagulated (EDTA) blood (8 mL) is drawn from an anesthetized monkey. ¹⁸F-labeled test compound (15 μCi) is added to whole blood (1.5 mL), which is then sampled (50 μL) at 0.5, 1, 2, 3, 4, 5, 10, 15, 30, 60, and 90 min. Each sample is added immediately to acetonitrile that has been spiked with test compound. The mixture is then agitated, diluted with water (50 μL), and mixed. The mixture is counted for radioactivity in a gamma-counter and centrifuged at 9400×g for 1 min. The clear supernatant liquid is analyzed with HPLC and the precipitate counted for radioactivity in a gamma-counter. The effect of sodium azide on the stability of [¹⁸F]test compound in whole monkey blood is also assessed. Whole blood (1 mL) is mixed well with a solution (20 μL) of sodium azide (6 mg). [¹⁸F]Test compound is added and the solution incubated at room temperature for 190 min before centrifugation at 1800×g for 4 min. The supernatant plasma is treated with acetonitrile and analyzed with HPLC.

The stability of ¹⁸F-labeled-test compound in monkey plasma is assessed as follows. Whole monkey blood (2.0 mL) is centrifuged at 1800×g for 5 min. Supernatant plasma (1.0 mL) is added to ¹⁸F-labeled test compound (5 μCi) and the solution incubated at RT for 168 min. A sample is then analyzed by HPLC.

To test the stability of ¹⁸F-labeled test compound in whole human blood, ¹⁸F-labeled test compound (2 μCi) is added to human whole blood (2.0 mL), incubated at 23° C., and sampled (50 μL) at 10, 15, 20, 30, 40, 60, and 90 min. Each sample is placed in acetonitrile (50 μL) that had been spiked with test compound and mixed well. Then potassium fluoride solution (0.5 M; 50 mL) is added to each sample before mixing again, counting for radioactivity in a c-counter, and centrifugation at 10,000×g for 1 min. Each supernatant liquid is injected onto HPLC for analysis. The stability of ¹⁸F-labeled test compound in aqueous solution is also checked. Thus, solutions of ¹⁸F-labeled test compound in water alone and in saline (0.9%) containing phosphoric acid (0.15 mM) are prepared and sampled at set times (34, 48, and 159 min for water solution and 54 and 71 min for salt solution) for immediate HPLC analysis.

### Example 9

### Stability of ¹⁸F-Lableled]Test Compound in Monkey and Human Brain Homogenates In Vitro

Monkey brain tissue (3.34 g), previously stored for 1 year at −70° C., is thawed slowly at room temperature and then homogenized with ¹⁸F-labeled test compound (about 100 μCi). The tissue suspension is ice-cooled between each pass of homogenization. Brain homogenate is sampled after incubation at 37° C. in an oscillating water bath at 5, 10, 15, 30, 60, and 90 min.

Human brain tissue (1.76 g), previously stored for a few weeks at −70° C., is thawed slowly at room temperature and then homogenized with ice-cold saline (3 mL). The tissue suspension is ice cooled between each pass of homogenization. ¹⁸F-labeled test compound (about 50 μCi) in ethanol-water (1:1 v/v; 450 μL) is slowly mixed into to the cold homogenate. This mixture is incubated at 37° C. in an oscillating water bath and sampled (50 μL) after 5, 10, 15, 30, 45, 60, 90, 120, and 180 min.

Samples from incubations of ¹⁸F-labeled test compound with monkey or human brain homogenate are added to acetonitrile (700 μL), which has been spiked with test compound and mixed well. Then potassium fluoride solution (0.5 M; 50 μL) is added, and the solution is mixed well again. Each sample is counted in a gamma-counter and centrifuged at 10,000×g for 1 min. The supernatant liquids are analyzed with HPLC. The precipitates are counted in a gamma-counter to enable calculation of the recovery of radioactivity into acetonitrile.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A compound of the formula 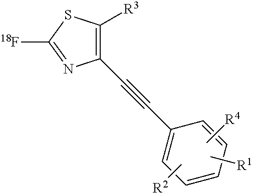
wherein:
R¹ is hydrogen, cyano, C₁-C₂alkylnitrile, hydroxyl, C₁-C₂alkyl, aryl, C₁-C₂alkoxy, halogen, trifluoromethyl, or trifluoromethoxy;
R² is hydrogen, cyano, C₁-C₂alkylnitrile, hydroxyl, C₁-C₂alkyl, aryl, C₁-C₂alkoxy, halogen, trifluoromethyl, or trifluoromethoxy;
R³ is hydrogen, halogen, methyl, or methoxy; and
R⁴ is 0 to 3 substituents independently chosen from halogen, methyl, and methoxy.

2. A compound of claim 1 of the formula:

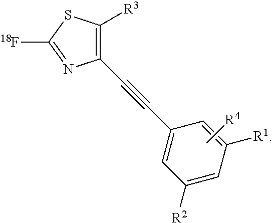

3. A compound of claim 2 wherein: R³ is hydrogen and R⁴ is 0 substituents.

4. A compound of claim 3 wherein: R¹ is hydrogen, halogen, or cyano; and R² is hydrogen, halogen, or cyano.

5. A compound of claim 4 wherein: R¹ is hydrogen or fluoro; and R² is hydrogen or cyano.

6. A compound of claim 4 wherein: R¹ and R² are both hydrogen.

7. A compound of claim 4 wherein: R¹ is hydrogen and R² is cyano.

8. A compound of claim 4 wherein: R¹ is fluoro and R² is cyano.

9. A method of making a compound of Formula II 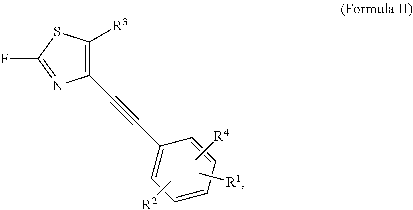
wherein
R¹ is hydrogen, cyano, C₁-C₂alkylnitrile, hydroxyl, C₁-C₂alkyl, aryl, C₁-C₂alkoxy, halogen, trifluoromethyl, or trifluoromethoxy;
R² is hydrogen, cyano, C₁-C₂alkylnitrile, hydroxyl, C₁-C₂alkyl, aryl, C₁-C₂alkoxy, halogen, trifluoromethyl, or trifluoromethoxy;
R³ is hydrogen, halogen, methyl, or methoxy; and
R⁴ is 0 to 3 substituents independently chosen from halogen, methyl, and methoxy;
comprising contacting a compound of Formula 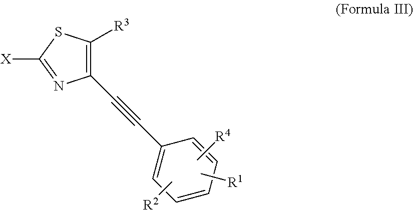
where X is a halogen radical selected from chloro, bromo, and iodo; or
X is nitro, trimethylammonium or ArI⁺, where Ar is phenyl or 2-thienyl, each of which Ar is unsubstituted or substituted with 1, 2, or 3 substituents independently chosen from halogen, C₁-C₂alkyl, and C₁-C₂alkoxy;
with KF, CsF or a KF-AgF mixture a non-polar organic solvent under thermal heating or microwave conditions to provide a compound of Formula II.

10. The method of claim 9 of making a compound of Formula IIA 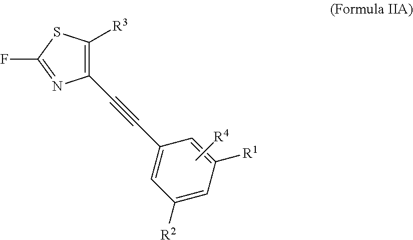
comprising contacting a compound of Formula IIIA 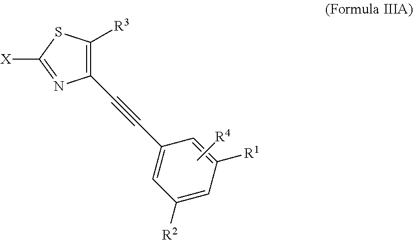
with KF, CsF or a KF-AgF mixture a non-polar organic solvent under thermal heating or microwave conditions to provide a compound of Formula IIA.

11. The method of claim 10, wherein Ar is phenyl, 2-thienyl, phenyl substituted with 1 or 2 methoxy substituents, or 2-thienyl substituted with 1 or 2 methyl substituents.

12. The method of claim 11 wherein
R¹ is hydrogen, halogen, or cyano; and R² is hydrogen, halogen, or cyano;
R³ is hydrogen and R⁴ is 0 substituents.

13. A method of making a compound of claim 1, comprising microwave irradiation or thermal heating of a compound of the formula 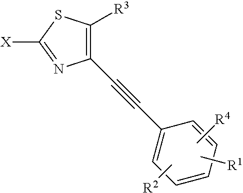
where X is a halogen radical selected from chloro, bromo, and iodo; or
X is nitro, trimethylammonium or ArI⁺, where Ar is phenyl or 2-thienyl, each of which Ar is unsubstituted or substituted with 1, 2, or 3 substituents independently
chosen from halogen, C₁-C₂alkyl, and C₁-C₂alkoxy;
with [¹⁸F]F⁻—K⁺-4,7,13,16,21,24-hexaoxa-1,10-diazabicyclo[8.8.8]hexacosane or 18-Crown-6 in a non-polar organic solvent to provide a compound of claim 1.

14. The method of claim 13, comprising microwave irradiation or thermal heating of a compound of the formula 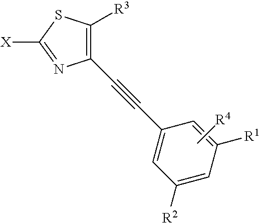
with [¹⁸F]F⁻—K⁺-4,7,13,16,21,24-hexaoxa-1,10-diazabicyclo[8.8.8]hexacosane or 18-crown-6 in a non-polar organic solvent to provide a compound of claim 1.

15. The method of claim 10 wherein the compound of formula 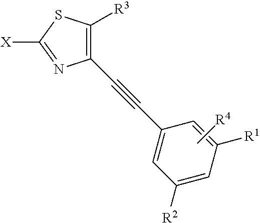
is provided by halogenating a compound of the formula 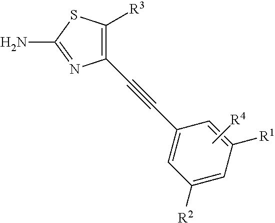
with
(i) CuX¹, where X¹ is chloro, bromo, or iodo, in the presence of n-butyl nitrite; or
(ii) alumina-KCuBr₂.

16. A method of imaging mGluR5 in a mammal comprising administering a compound of claim 1 to the mammal and imaging portions of the mammal where mGluR5 occurs.

17. The method of claim 16 wherein the mammal is a rat, a monkey, or a human.

18. A method of imaging mGluR5 in vitro comprising
contacting a sample containing mGluR5 with a compound of claim 1, removing the unbound compound from the sample, and
detecting the bound compound in the sample.

19. The method of claim 18 wherein the sample is a brain section, and the detecting is autoradiography.

20. A method of treating an mGluR5 modulated disorder in a patient having an mGluR5 mediated disorder comprising providing a therapeutically effective amount of a compound of the formula 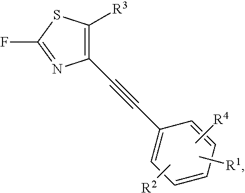
to the patient, wherein
R¹ is hydrogen, cyano, C₁-C₂alkylnitrile, hydroxyl, C₁-C₂alkyl, aryl, C₁-C₂alkoxy, halogen, trifluoromethyl, or trifluoromethoxy;
R² is hydrogen, cyano, C₁-C₂alkylnitrile, hydroxyl, C₁-C₂alkyl, aryl, C₁-C₂alkoxy, halogen, trifluoromethyl, or trifluoromethoxy;
R³ is hydrogen, halogen, methyl, or methoxy; and
R⁴ is 0 to 3 substituents independently chosen from halogen, methyl, and methoxy.

21. The method of claim 20 comprising providing a therapeutically effective amount of a compound of the formula

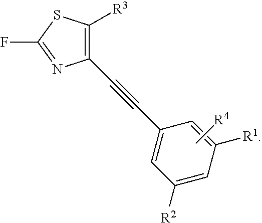

22. The method of claim 21, wherein
R¹ is hydrogen, halogen, or cyano; and R² is hydrogen, halogen, or cyano;
R³ is hydrogen and R⁴ is 0 substituents.

23. The method of claim 21, wherein the mGluR5 mediated disorder is schizophrenia, Alzheimer's disease, anxiety, depression, drug addiction, or fragile X syndrome.
